(19) United States
(12) Patent Application Publication
(10) Pub. No.: US 2013/0116560 A1
(43) Pub. Date: May 9, 2013

(54) ULTRASOUND TEMPERATURE MAPPING SYSTEM AND METHOD

(71) Applicants: Wen-Shiang CHEN, Taipei (TW); Der-Hsien Lien, Taipei (TW); Chuin-Shan Chen, Taipei (TW); Jay Shieh, Taipei (TW); Chiung-Nien Chen, Taipei (TW); Chien-Cheng Chang, Taipei (TW); Yu-Chen Shu, Taipei (TW); Chang-Wei Huang, Taipei (TW)

(72) Inventors: Wen-Shiang CHEN, Taipei (TW); Der-Hsien Lien, Taipei (TW); Chuin-Shan Chen, Taipei (TW); Jay Shieh, Taipei (TW); Chiung-Nien Chen, Taipei (TW); Chien-Cheng Chang, Taipei (TW); Yu-Chen Shu, Taipei (TW); Chang-Wei Huang, Taipei (TW)

(73) Assignee: National Taiwan University, Taipei City (TW)

(21) Appl. No.: 13/418,702

(22) Filed: Mar. 13, 2012

(30) Foreign Application Priority Data
Nov. 3, 2011 (TW) 100140071

(51) Int. Cl. A61B 5/01; A61B 8/13

(52) U.S. Cl. 600/438

(57) ABSTRACT

The present application relates to an ultrasound temperature mapping system and method. The ultrasound temperature mapping system for measuring a temperature of an object comprises an ultrasound transducer and a processing module. The ultrasound transducer is configured to acquire a first image and a second image with respect to the object. The processing module implements a zero-crossing algorithm to process the first image to yield a plurality of first zero-crossing points and implements a cross-correlation algorithm to process the first image and the second images based on the plurality of first zero-crossing points so as to obtain a plurality of displacements. The processing module further calculates the temperature based on the plurality of displacements.

### BACKGROUND OF THE INVENTION

1. Field of the Invention

The present invention relates to a temperature mapping system and method, and more particularly, to an ultrasound temperature mapping system and method.

2. Description of the Prior Art

Currently, non-invasive therapies have been highly valued in clinical practice. Among the non-invasive therapies, thermal therapy is widely applied for the control of cancer cells, tissue ablation, etc. Therefore, non-invasive operation has become the most distinctive feature of the ultrasound thermal therapy.

During the thermal therapy process, a mapping system capable of showing real-time local temperature changes is very important as it enables the user to monitor the level of heating to prevent damages to the surrounding normal tissues. Clinicians will be unable to know precise temperature changes within internal tissues without such monitoring systems, and this increases not only the difficulty in performing the treatment, but also the risk during the operation process, restricting the application of thermal therapy in clinical practice.

Known measurement techniques include internal impedance temperature measurement, MRI, infrared temperature measurement, ultrasound tissue temperature estimation, etc. These techniques are used to measure and monitor the temperature of the tissue. However, each of these techniques has limitations. For example, the internal impedance temperature measurement is disadvantageous in that it has a low spatial resolution and a high degree of variation and is less frequently used in clinical practice. Though MRI features a higher spatial resolution, its ability to obtain real-time measurements is limited by the extremely slow scanning speed. Moreover, the MRI system is expensive and bulky and thus cannot be readily integrated with other temperature therapies. The infrared temperature measurement is incapable of showing temperature changes in deep tissues and thus not suitable for use as a temperature monitoring equipment during the thermal therapy process.

The conventional technique employs ultrasound to obtain temperature distribution information and is characterized by non-invasive measurement, instant image scanning, good system mobility, low cost, etc. However, the fundamental limitation of such technique is that the accuracy of the information obtained is not satisfactory.

Therefore, a need exists in the art for a system and method that employ ultrasound to obtain temperature distribution information while increasing the accuracy of the temperature distribution information.

### SUMMARY OF THE INVENTION MODEL

The present application relates to an ultrasound temperature mapping system and method that combine the advantages of cross-correlation algorithm and zero-crossing algorithm to improve the accuracy of ultrasound temperature measurements.

The present invention provides an ultrasound temperature mapping system for measuring a temperature of an object, comprising: an ultrasound transducer and a processing module. The ultrasound transducer is configured to acquire a first image and a second image with respect to the object. The processing module implements a zero-crossing algorithm to process the first image to yield a plurality of first zero-crossing points and implements the cross-correlation algorithm to process the first image and the second image based on the plurality of first zero-crossing points so as to obtain a plurality of displacements. The processing module further calculates the temperature based on the plurality of displacements.

The present invention provides an ultrasound temperature mapping method for measuring a temperature of an object, comprising: acquiring a first image and a second image with respect to the object; implementing a zero-crossing algorithm to process the first image to yield a plurality of first zero-crossing points; implementing a cross-correlation algorithm to process the first image and the second image based on the plurality of first zero-crossing points so as to obtain a plurality of displacements; and calculating a temperature based on the plurality of displacements.

The present invention will be described by way of a preferred, embodiment and the accompanying drawings so as to facilitate the understanding of the aforementioned contents.

### BRIEF DESCRIPTION OF THE DRAWINGS

FIG. 1 is a diagram illustrating the configuration of an ultrasound temperature mapping system in accordance with one embodiment of the present invention.

FIG. 2 is a flow chart illustrating an ultrasound temperature mapping method in accordance with one embodiment of the present invention.

FIG. 3 is a diagram illustrating the relation between the amplitude and depth of the acquired data with respect to the object S.

FIG. 4 is a diagram illustrating the intersection of the first and second data M1 and M2 and the X axis.

FIG. 5 is a flow chart illustrating a method for increasing the accuracy in accordance with one embodiment of the present invention.

FIG. 6 illustrates the relation between displacements and temperature changes with respect to the unheated object, the once-heated object and the twice-heated object, respectively.

FIG. 7 illustrates the displacements calculated using CCR, ZCT and ZCT+FCCR, respectively.

### DETAILED DESCRIPTION OF THE PREFERRED EMBODIMENTS

While the present invention will be fully described with preferred embodiments, it is to be understood beforehand that those skilled in the art can make modification to the invention described and attain the same effect, and that the description below is a general representation to those skilled in the art and is not intended to limit the scope of the present invention.

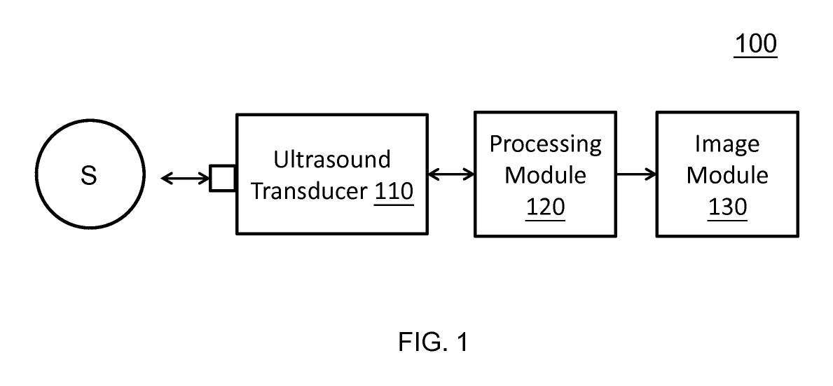

FIG. 1 is a diagram illustrating the configuration of an ultrasound temperature mapping system 100 in accordance with one embodiment of the present invention. In the embodiment of the present invention, the ultrasound temperature mapping system 100 comprises at least one ultrasound transducer 110 and a processing module 120. In one embodiment, the ultrasound temperature mapping system 100 may further comprise an image module 130.

In one embodiment, the ultrasound transducer 110, e.g. a focused ultrasound transducer for measurement, may be driven by an ultrasound pulse generator to emit an ultrasound signal to an object S and receive a reflected echo signal. That is, the ultrasound transducer 110 can be used to acquire images with respect to the object S.

The processing module 120, e.g. a microprocessor, is configured to process the received signal. In one embodiment, the processing module 120 may further comprise a storage device storing algorithms, such as the cross-correlation algorithm and the zero-crossing algorithm, so as to calculate the received signal with the algorithms stored in the storage device. In another embodiment, algorithms such as the cross-correlation algorithm and the zero-crossing algorithm can be embodied in form of hardware and implemented by the processing module 120 to accelerate the calculation speed.

The image module 130, e.g. a display screen, is configured to display images, such as images about temperatures of an object, for the user. In one embodiment, the image module 130 may be, for example, a projection module for projecting images onto a plane.

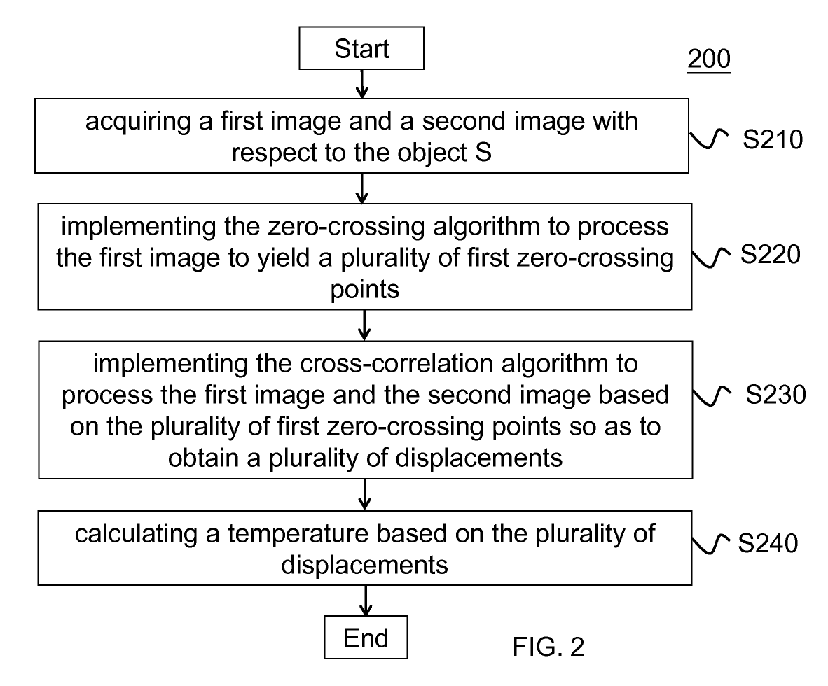

FIG. 2 is a flow chart illustrating an ultrasound temperature mapping method 200 for measuring a temperature of an object S in accordance with one embodiment of the present invention. Please also refer to FIG. 1.

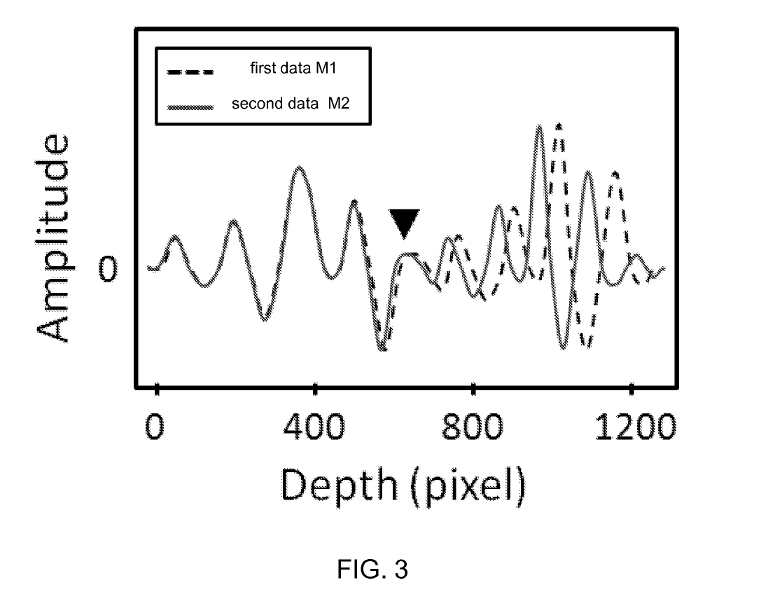

In S210, the ultrasound transducer 110 acquires a first image and a second image with respect to the object S. For example, the ultrasound temperature mapping system 100 is used to detect the temperature of the object S. As the temperature of the object S increases, the data of the first and second images acquired by the ultrasound transducer 110 are different. FIG. 3 is a diagram illustrating the relation between the amplitude and depth of the acquired data with respect to the object S. A first data and a second data, e.g. M1 and M2 shown in FIG. 3, are obtained by using the processing module 120 to process the first and second images. In one embodiment, the first image may be acquired before the temperature of the object S changes, and the second image may be acquired after the temperature of the object S changes.

When the two data acquired before and after the temperature change occurs are compared, it is clear that signal delay occurs at a position where the temperature starts to rise, for example the symbol ▾ in FIG. 3. The delay occurs because the transmission speed of sound waves within a medium varies with the temperature. Generally, sound waves are transmitted faster in areas of a higher temperature within a medium.

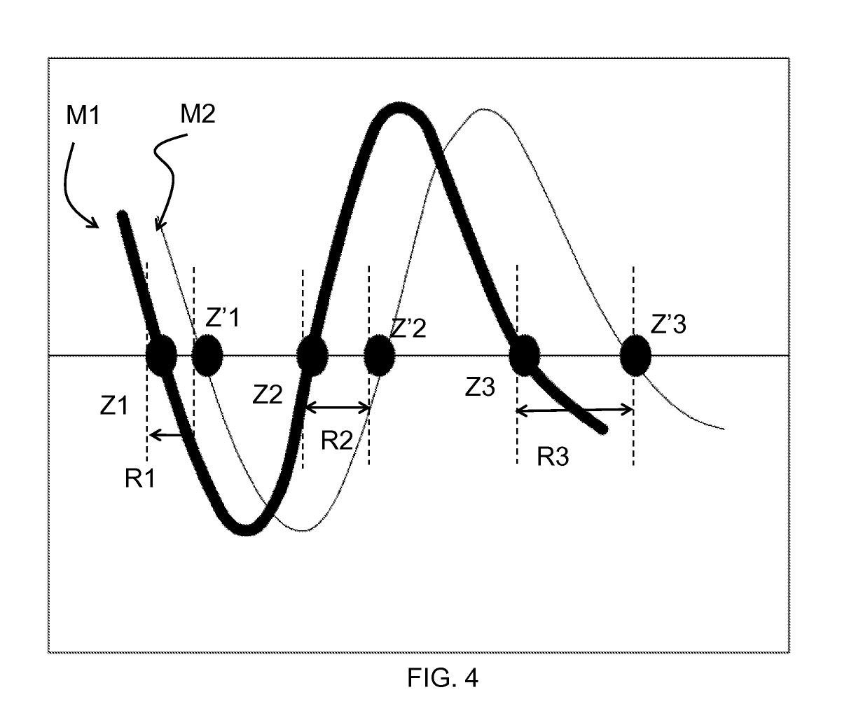

In S220, the processing module 120 implements the zero-crossing algorithm to process the first image to yield a plurality of first zero-crossing points. FIG. 4 is a diagram illustrating the intersection of the first and second data M1 and M2 and the X axis. For example, the first data and the second data are obtained by processing the first and second images with the processing module 120. In one embodiment, for example, the processing module 120 processes the first image to obtain the first data M1 and then implements the zero-crossing algorithm to process the first data M1 to yield a plurality of first zero-crossing points, e.g. Z1, Z2 and Z3 shown in FIG. 4. While three zero-crossing points are used in the example, the present invention is not limited thereto. Any zero-crossing point yielded based on the first image falls within the scope of the present invention.

In S230, the processing module 120 implements the cross-correlation algorithm to process the first image and the second image based on the plurality of first zero-crossing points so as to obtain a plurality of displacements. For example, referring to FIG. 4, the first data M1 is obtained based on the first image, and the processing module 120 can implement the cross-correlation algorithm to process respective areas of the first zero-crossing points Z1, Z2 and Z3 of the first image and the corresponding areas of zero-crossing points obtained based on the second data M2 of the second image. Consequently, a plurality of displacements, e.g. D1, D2 and D3, can be obtained.

In one embodiment, the cross-correlation algorithm is implemented to calculate the plurality of displacements based on values within a specific range adjacent to the first zero-crossing points Z1-Z3 of the first data M1 and values within a specific range corresponding to the first zero-crossing points Z1-Z3 of the second image. For example, the specific range covers 25 pixels. For example, the value within 25 pixels adjacent to the first zero-crossing point Z1 is R1, and the processing module 120 can implement the cross-correlation algorithm to calculate the displacements of the first image and the second image within R1. The displacements within R2 and R3 can be calculated in the same way. While 25 pixels are used in the example, the present invention is not limited thereto. Any specific range, such as a first value (for example, the previous 5 pixels) before the zero-crossing point and a second value (for example the following 45 pixels) after the zero-crossing point, falls within the scope of the present invention.

In S240, the processing module 120 calculates a temperature based on the plurality of displacements. For example, as the displacements calculated by the ultrasound temperature mapping system 100 correspond to variations in the sound speed, the processing module 120 can derive the variances in temperature based on the relative relation between the transmission speed of sound waves and the temperature of the medium, thereby the temperature of the object S can be derived.

However, each of the cross-correlation algorithm and the zero-crossing algorithm has limitations. For example, the cross-correlation algorithm employs the similarity between two data to calculate the relative displacement. If the similarity between two signals is degraded by noise in the local area, a miscalculation may occur. Miscalculation also occurs when another similar location is matched. As the ultrasound signal is similar to the sinusoidal wave signal, an incorrect area may be matched if the characteristics of signals in the area to be matched are not distinctive enough. Moreover, another disadvantage of the cross-correlation algorithm is that the calculation load is heavy and thus the time required for calculation is longer. However, the cross-correlation algorithm is characterized by more accurate matching.

The zero-crossing algorithm employs the passing of two signals through the X axis to calculate the displacement. Ideally, the number of zero-crossing points of the two signals passing through the X axis is the same, and the displacement of each area can be obtained by one-to-one matching. But, in real situation, the number of zero-crossing points of the two signals will not be the same and zero-crossing point missing occurs often. When the displacement is greater than a cycle of a sinusoidal wave, the matching of zero-crossing points will be more difficult. In other words, the zero-crossing algorithm may not be applicable at an extremely high temperature where the displacement becomes large.

The ultrasound temperature mapping system and method of the present invention combine the advantages of the cross-correlation algorithm and the zero-crossing algorithm and characterized by simple computation of the zero-crossing algorithm and more accurate matching results of the cross-correlation algorithm.

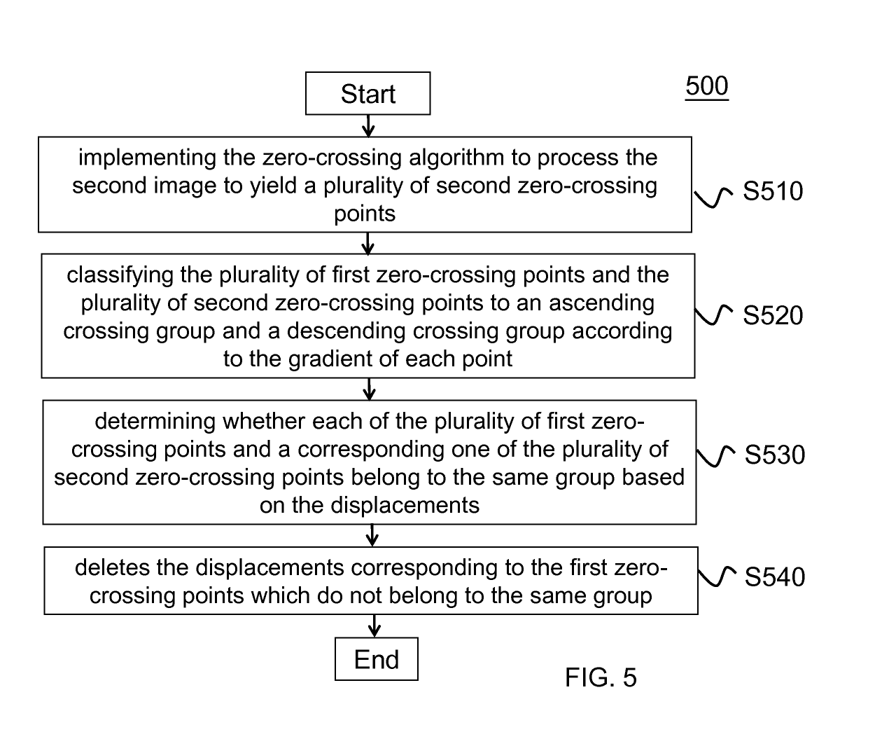

In one embodiment, the ultrasound temperature mapping method illustrated in FIG. 2 may further comprise steps of increasing the accuracy. FIG. 5 is a flow chart illustrating a method 500 for increasing the accuracy in accordance with one embodiment of the present invention. In S510, the processing module 120 implements the zero-crossing algorithm to process the second image to yield a plurality of second zero-crossing points. For example, referring to FIG. 4, the processing module 120 processes the second image to obtain a second data M2 and yield a plurality of second zero-crossing points Z′1-Z′3.

In S520, the processing module 120 classifies the plurality of first zero-crossing points Z1-Z3 and the plurality of second zero-crossing points Z′1-Z′3 to an ascending crossing group and a descending crossing group according to the gradient of each point. For example, the amplitudes of M1 and M2 transit from positive amplitudes to negative amplitudes at Z1 and Z′1 on the X axis, thus Z1 and Z′1 have negative gradients and are classified to the descending crossing group. Z3 and Z′3 are classified to the descending crossing group for the same reason. As the amplitudes of M1 and M2 transit from negative amplitudes to positive amplitudes at Z2 and Z′2, they are classified to the ascending crossing group.

In S530, the processing module 120 determines whether each of the plurality of first zero-crossing points and a corresponding one of the plurality of second zero-crossing points belong to the same group based on the displacements.

In S540, the processing module 120 deletes the displacements corresponding to the first zero-crossing points which do not belong to the same group.

For example, the plurality of first zero-crossing points Z1-Z3 added with respective displacements are supposed to correspond to the plurality of second zero-crossing points Z′1-Z′3. However, the waveform of the data actually obtained may not be as perfect as those shown in FIGS. 3 and 4 and may have many crossing points on the X axis due to the interference of noise. If the second zero-crossing point to which the first zero-crossing point added with the displacement corresponds and the first zero-crossing point it actually corresponds to do not belong to the same group, it is likely that this first zero-crossing point is not the correct crossing point but a point intersecting the X axis due to the interference of noise.

In the method illustrated in FIG. 5, as the displacements of incorrect crossing points can be deleted, the interference of noise is reduced, thereby increasing the accuracy of temperature estimations.

In another embodiment integrated with the method illustrated in FIG. 5, the processing module 120 can further calculate a median of the plurality of displacements and delete the displacement having a great disparity with the median. For example, if the median of the plurality of displacements calculated by the processing module 120 is 5, and a displacement, e.g. 15, has a great disparity with the adjacent displacements as well as the median, the processing module 120 can delete this displacement apparently affected by noise so as to ensure the accuracy of temperature estimations.

After the displacement is calculated, e.g. after S240, whether or not the steps of increasing the accuracy, such as the aforementioned method employing a median or the method illustrated in FIG. 5, are implemented or parts of the inaccurate displacements are deleted, the processing module 120 can implement an interpolation to calculate the displacement of each point of the second image with respect to the first image based on the remaining displacements.

After the displacement of each point of the second image with respect to the first image is calculated, the computation module 120 can perform a derivative operation on the displacement of each point to obtain the temperature of the object S and the position where the temperature change occurs.

For example, referring to FIG. 3, if the displacement calculated based on the first zero-crossing point Z2 is not accurate, e.g. the displacement has a great disparity with the median, the displacement calculated based on the first zero-crossing point Z2 will be deleted. The displacement of each point between the first zero-crossing point Z1 and the first zero-crossing point Z3 can be obtained by performing the interpolation on the basis of the displacement calculated based on the first zero-crossing point Z1 and the displacement calculated based on the first zero-crossing point Z3.

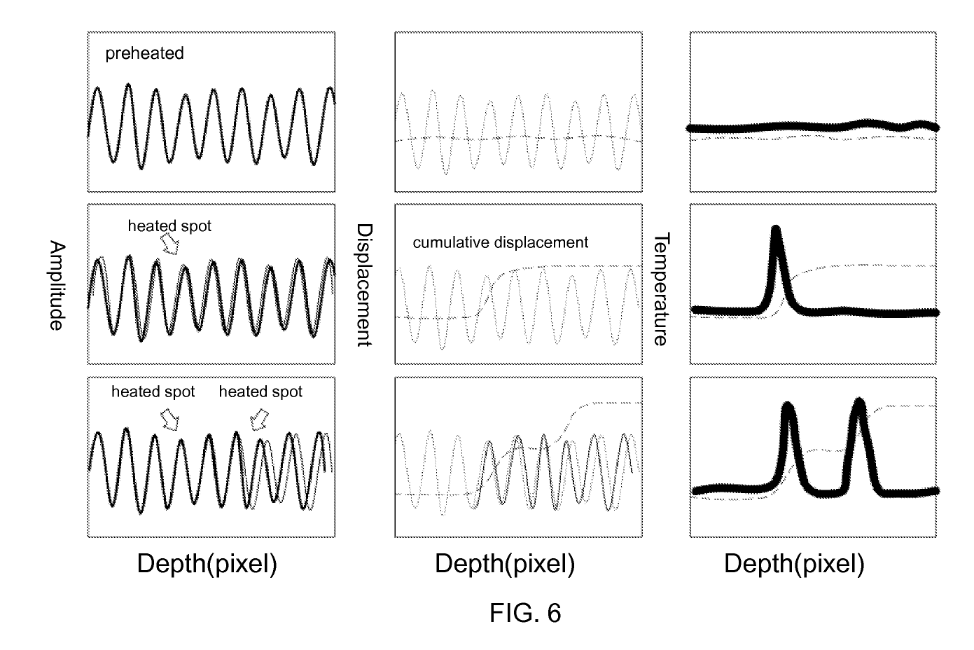

Regarding the obtainment of the temperature of the object S and the position where the temperature change occurs, for example, please refer to FIG. 6 which illustrates the relation between displacements and temperature changes with respect to the unheated object, the once-heated object and the twice-heated object, respectively. The first diagram of the leftmost section shows the data obtained by processing the image of the unheated object acquired by the ultrasound transducer 110 with the processing module 120. The second diagram shows the data obtained by processing the image of the once-heated object acquired by the ultrasound transducer 110 with the processing module 120. The third diagram shows the data obtained by processing the image of the twice-heated object acquired by the ultrasound transducer 110 with the processing module 120.

The displacements processed with the ultrasound temperature mapping system 100 and the method thereof are shown in the three diagrams on the middle section of FIG. 6. The three diagrams show the displacements with respect to the unheated object S, the once-heated object and the twice-heated object, respectively. It can be seen from the first diagram on the middle section of FIG. 6 that there is no displacement because the object S is not heated. Under the circumstance that the object S is heated once, when the displacement of each point accumulates to certain level after the heated spot, the cumulative displacement of the pixels will remain at the accumulated level, as shown in the second diagram on the middle section of FIG. 6. When the object S is heated twice, the displacement will uprise twice corresponding to the heated spots, as shown in the third diagram on the middle section of FIG. 6.

The variances in temperature and the positions where temperature changes occur can be derived by performing derivative operation on the displacements, as shown in the three diagrams on the rightmost section of FIG. 6.

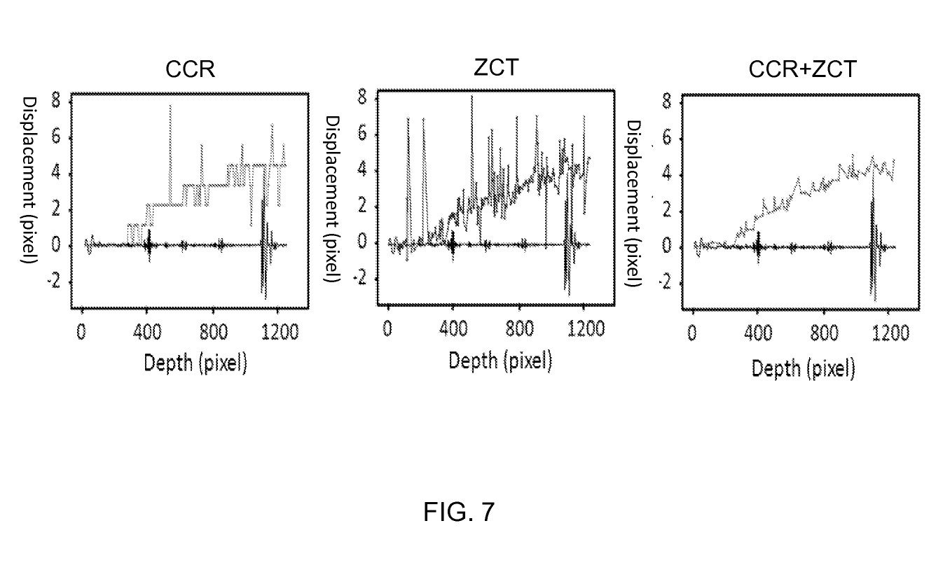

In conclusion, the ultrasound temperature mapping system and method of the present invention combine the advantages of the cross-correlation algorithm and the zero-crossing algorithm so that the temperature estimations are more accurate. FIG. 7 illustrates the displacements calculated using CCR, ZCT and ZCT+CCR, respectively. It can be seen from FIG. 7 that there are more errors in the displacements calculated exclusively using CCR or ZCT and that the curve representing displacements calculated using ZCT+CCR is more smooth.

While this invention has been described by way of a preferred embodiment, it is to be understood that this invention is not limited hereto. A person having ordinary skill in the art can make various changes and alterations herein without departing from the spirit and scope of this invention. The scope of protection of the present invention is defined by the appended claims.

What is claimed is:

1. An ultrasound temperature mapping system for measuring a temperature of an object, comprising:
an ultrasound transducer configured to acquire a first image and a second image with respect to the object; and
a processing module implementing a zero-crossing algorithm to process the first image to yield a plurality of first zero-crossing points and implementing a cross-correlation algorithm to process the first image and the second image based on the plurality of first zero-crossing points so as to obtain a plurality of displacements;
wherein the processing module further calculates the temperature based on the plurality of displacements.

2. The temperature mapping system according to claim 1, wherein the first image is acquired before the temperature of the object changes and the second image is acquired after the temperature of the object changes.

3. The temperature mapping system according to claim 1, wherein the cross-correlation algorithm is implemented to calculate the plurality of displacements based on values within a specific range adjacent to the plurality of first zero-crossing points of the first image and values within the specific range corresponding to the plurality of first zero-crossing points of the second image.

4. The temperature mapping system according to claim 1, wherein the processing module further implements the zero-crossing algorithm to process the second image to yield a plurality of second zero-crossing points, classifies the plurality of first zero-crossing points and the plurality of second zero-crossing points to an ascending crossing group and a descending crossing group according to a gradient of each point, determines whether each of the plurality of first zero-crossing points and a corresponding one of the plurality of second zero-crossing points belong to the same group based on the displacements, and deletes the displacements corresponding to the first zero-crossing points which do not belong to the same group.

5. The temperature mapping system according to claim 4, wherein the processing module performs an interpolation to calculate a displacement of each point of the second image with respect to the first image based on the remaining displacements.

6. The temperature mapping system according to claim 5, wherein the processing module performs a derivative operation on the displacement of each point to obtain the temperature, and wherein the temperature mapping system further comprises:
an image module displaying the temperature.

7. The temperature mapping system according to claim 1, wherein the processing module further calculates a median of the plurality of displacements and deletes a displacement having a great disparity with the median.

8. The temperature mapping system according to claim 7, wherein the processing module performs an interpolation to calculate a displacement of each point of the second image with respect to the first image based on the remaining displacements.

9. The temperature mapping system according to claim 8, wherein the processing module performs a derivative operation on the displacement of each point to obtain the temperature, and wherein the temperature mapping system further comprises:
an image module displaying the temperature.

10. An ultrasound temperature mapping method for measuring a temperature of an object, comprising:
acquiring a first image and a second image with respect to the object;
implementing a zero-crossing algorithm to process the first image to yield a plurality of first zero-crossing points;
implementing a cross-correlation algorithm to process the first image and the second image based on the plurality of first zero-crossing points so as to obtain a plurality of displacements; and
calculating a temperature based on the plurality of displacements.

11. The temperature mapping method according to claim 10, wherein the cross-correlation algorithm is implemented to calculate the plurality of displacements based on values within a specific range adjacent to the plurality of first zero-crossing points of the first image and values within the specific range corresponding to the first zero-crossing points of the second image.

12. The temperature mapping method according to claim 11, wherein the specific range covers 25 pixels.

13. The temperature mapping method according to claim 10 further comprising:
implementing the zero-crossing algorithm to process the second image to yield a plurality of second zero-crossing points;
classifying the plurality of first zero-crossing points and the plurality of second zero-crossing points to an ascending crossing group and a descending crossing group according to a gradient of each point;
determining whether each of the plurality of first zero-crossing points and a corresponding one of the plurality of second zero-crossing points belong to the same group based on the displacements; and
deleting the displacements corresponding to the first zero-crossing points which do not belong to the same group.

14. The temperature mapping method according to claim 13 further comprising:
performing an interpolation to calculate a displacement of each point of the second image with respect to the first image.

15. The temperature mapping method according to claim 14 further comprising:
performing a derivative operation on the displacement of each point to obtain the temperature.

16. The temperature mapping method according to claim 10 further comprising:
calculating a median of the plurality of displacements; and
deleting a displacement having a great disparity with the median.

17. The temperature mapping method according to claim 16 further comprising:
performing an interpolation to calculate a displacement of each point of the second image with respect to the first image.

18. The temperature mapping method according to claim 17 further comprising:
performing a derivative operation on the displacement of each point to obtain the temperature.

19. The temperature mapping method according to claim 10, wherein the first image is acquired before the temperature of the object changes and the second image is acquired after the temperature of the object changes.
